(19) United States
(12) Patent Application Publication
(10) Pub. No.: US 2021/0121096 A1
(43) Pub. Date: Apr. 29, 2021

(54) RESPIRATORY FAILURE DETECTION SYSTEMS AND ASSOCIATED METHODS

(71) Applicant: University of Washington, Seattle, WA (US)

(72) Inventors: Shyamnath Gollakota, Seattle, WA (US); Jacob Sunshine, Seattle, WA (US); Rajalakshmi Nandakumar, Seattle, WA (US)

(21) Appl. No.: 17/051,745

(22) PCT Filed: May 23, 2019
(86) PCT No.: PCT/US19/33852

§ 371 Date: Oct. 29, 2020

Related U.S. Application Data
(60) Provisional application No. 62/675,560, filed on May 23, 2018.

(51) Int. Cl. A61B 5/08; A61B 5/00; A61B 5/113

(52) U.S. Cl. CPC A61B 5/0816; A61B 5/7282; A61B 5/113; A61B 5/746; A61B 5/7246

(57) ABSTRACT

An respirator}′ failure detection system and associated devices and methods are disclosed herein. In one embodiment, one or more transducers of a mobile device emit acoustic energy toward a subject and acquire a corresponding reflected signal. In some embodiments, the system analyzes the reflected signal to determine a distance between the subject and the mobile device. The system extracts motion data of the subject from the reflected signal. Based at least in part on the extracted motion data, the system identifies gross motor motion of the subject and/or determines one or more breathing parameters of the subject. In some embodiments, the system uses the breathing parameters to determine whether the subject is currently in need of rescue intervention. When the subject is currently in need of rescue intervention, the system can solicit help from emergency services, contact an emergency contact specified by the subject, and/or administer an antidote.

### CROSS-REFERENCE TO RELATED APPLICATION(S)

This application claims the benefit of U.S. Provisional Patent Application No. 62/675,560, filed May 23, 2018, which is incorporated herein by reference in its entirety.

### TECHNICAL FIELD

The present disclosure is related to systems and associated methods for monitoring a subject's motion and breathing. In particular, the present disclosure is directed to systems and methods for monitoring motion of a subject's body to identify events indicating respiratory failure.

### BACKGROUND

At high doses, opioids (particularly fentanyl) can cause rapid cessation of breathing (apnoea), hypoxemic/hypercarbic respiratory failure, and death, the physiologic sequence by which people commonly succumb from unintentional opioid overdose. Fatal opioid overdose remains a public health epidemic in the United States. Each day, over 100 people die from opioid overdose in the United States alone, and data from the Centers for Disease Control and Prevention (CDC) indicate the epidemic is worsening.

Unlike many life-threatening medical emergencies, opioid toxicity is readily reversed with rapid identification and administration of the overdose antidote (naloxone) or supportive respiratory care. Thus, a fundamental challenge of fatal opioid overdose events is that victims die alone or among untrained or impaired bystanders. Further, in many such cases, the victims receive no assistance or insufficient diagnosis and treatment. For example, historical 9-1-1 response times and locations of fatal opioid overdose events in Seattle, King County (geographic area of 5,975 km²) indicate that nearly 90% of fatal overdose instances occurred in locations reachable in an average 9-1-1 response time of under eight minutes. Thus, timely and accurate detection of opioid overdose events and rapid intervention within this response time can prevent future deaths from opioid overdose.

### BRIEF DESCRIPTION OF THE DRAWINGS

FIG. 1 is a schematic diagram of an opioid overdose detection system configured in accordance with various embodiments of the present technology.

FIG. 2 is a block diagram of a system configured in accordance with various embodiments of the present technology.

FIG. 3 is a flow diagram of a method of operating an opioid overdose detection system configured in accordance with various embodiments of the present technology.

FIG. 4 is a graph depicting a reflected signal acquisition approach in accordance with various embodiments of the present technology.

FIG. 5 is a graph illustrating a motion waveform extracted from a frequency bin of a primary transform of a reflected signal in accordance with various embodiments of the present technology.

FIG. 6 is a graph illustrating peaks identified in the motion waveform of FIG. 5 in accordance with various embodiments of the present technology.

FIG. 7 is a flow diagram of a method for detecting motion data in and constructing a motion waveform from a reflected audio signal in accordance with various embodiments of the present technology.

FIG. 8 is a flow diagram of a method for identifying events indicating a potentially fatal opioid overdose absent intervention in accordance with various embodiments of the present technology.

FIG. 9 is a graph illustrating a motion waveform depicting several central apnea opioid overdose events identified in accordance with various embodiments of the present technology.

FIG. 10 is a graph illustrating a motion waveform depicting a respiratory depression opioid overdose event identified in accordance with various embodiments of the present technology.

### DETAILED DESCRIPTION

The present technology relates generally to systems, devices, and associated methods for monitoring a subject's motion and breathing. In one embodiment of the present technology, the systems and methods monitor a subject's motion and breathing while the subject uses opioids. In these and other embodiments, the systems, devices, and methods transmit an inaudible acoustic signal toward the subject using a speaker. The acoustic signals reflect off a surface, such as a chest or abdomen of the subject, and return to a microphone after a time delay corresponding to the distance of the subject's chest or abdomen from the speaker/microphone. When the subject's chest or abdomen moves due to the subject's breathing, the distance between the subject's chest or abdomen and the speaker/microphone changes, resulting in a change in the time delay between when an acoustic signal is transmitted by the speaker and when it is received at the microphone as a reflected signal. In embodiments where the frequency of transmitted acoustic signals increases linearly over time, the time delays between when an acoustic signal of a frequency is transmitted by the speaker and when a reflected acoustic signal of the same frequency is received at the microphone translates to a unique frequency shift.

These frequency shifts are used to monitor the subject by measuring the changing distances between the subject's chest or abdomen and the speaker/microphone and to monitor the subject. For example, the systems, devices, and methods (e.g., continuously) monitor a distance of the subject from a measurement device by taking a primary transform (e.g., a fast Fourier transform) of the reflected signal and searching the resulting frequency bins of the transform for motion data related to the subject. In some embodiments, the systems, devices, and methods extract and analyze the motion data to detect gross motor motion of the subject. In these and other embodiments, the systems, devices, and methods extract and analyze the motion data to determine one or more breathing parameters (e.g., respiratory rate) of the subject. The systems, devices, and methods compare the calculated breathing parameters to one or more baseline breathing parameters of the subject and/or to one or more predetermined thresholds. Based at least in part on the comparison, the systems, devices, and methods can detect events (e.g., respiratory depression events, central apnea events, etc.) that indicate a potentially fatal opioid overdose absent intervention. In these and other embodiments, the systems, devices, and methods can trigger one or more alerts or alarms, solicit rescue intervention, and/or administer an opioid antidote to the subject in response to detecting events.

In several embodiments, systems of the present technology include a mobile device (e.g., a smartphone). The mobile device is configured to execute, at least in part, one or more methods of the present technology. For example, the mobile device in some embodiments is operated as a short-range active sonar, using frequency shifts in inaudible acoustic signals to identify respiratory depression, apnea, and gross motor movements associated with acute opioid toxicity. As mobile devices are largely ubiquitous, the systems, devices, and methods of the present technology obviate conventional, human-based approaches to overdose diagnosis that rely on medical grade equipment or trained recognition of diagnostic signs of opioid toxicity. As a result, the disclosed technology can reduce or eliminate the time and/or expenses associated with a technician monitoring a subject (e.g., in a hospital, in a self-injection facility, and/or in the subject's home, hotel room, or other location). Furthermore, in some embodiments, the disclosed technology provides concurrent monitoring and movement detection of multiple subjects via a single system and/or mobile device. In this manner, the present technology provides non-invasive, low-barrier, and harm reduction monitoring and/or rescue intervention for opioid or other drug users.

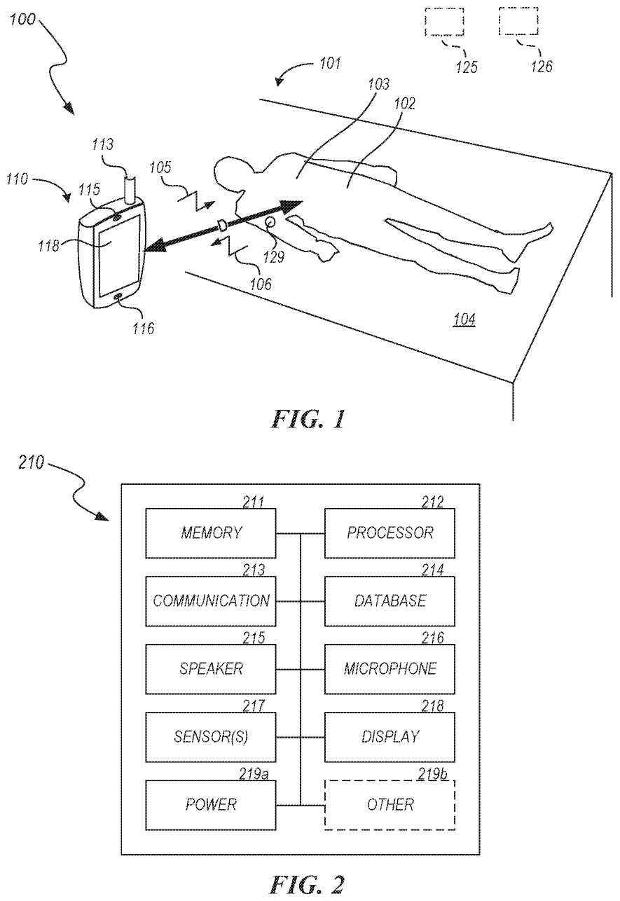

These and other aspects of the present disclosure are described in greater detail below. Certain details are set forth in the following description and in FIGS. 1-10 to provide a thorough understanding of various embodiments of the disclosure. Other details describing well-known systems and methods have not been set forth in the following disclosure to avoid unnecessarily obscuring the description of the various embodiments.

Although the present technology is primarily described below in the context of detecting opioid overdose, the present technology described herein may be used in a variety of applications, as will be appreciated by one skilled in the art. For example, the systems, devices, and methods of the present technology can be employed in the context of medical patient monitoring (e.g., in a hospital, in a patient's home, during post-surgery recovery, etc.) and/or to provide harm reduction monitoring and/or intervention to users of drugs other than opioids. For example, the systems, devices, and methods of the present technology can be employed to monitor a subject who uses benzodiazepines, alcohol, sleeping aid medications (e.g., ambien), anti-convulsant medication (e.g., gabapentin), and/or other drugs or medications in addition to or in lieu of opioids. The following select embodiments of the present technology are intended for illustrative purposes and do not limit the present technology to such applications.

### A. Select Embodiments of the Present Technology

FIG. 1 is a schematic diagram of an opioid overdose system 100 configured in accordance with various embodiments of the present technology. A device 110 of the system 100 is positioned near a subject 101 such that the subject's chest and abdomen 103 are approximately a distance D (e.g., 1 meter) from the device 110. A first transducer 115 (e.g., a loudspeaker) is configured to emit acoustic energy (e.g., sounds between about 20 Hz and 22 kHz or higher), including sound 105. A second transducer 116 (e.g., a microphone) is configured to receive acoustic energy including reflected sound 106 received from the subject's body 102. A communication link 113 (e.g., an antenna) communicatively couples the device 110 to a communication network (e.g., the Internet, a cellular telecommunications network, a WiFi network, etc.). A user interface 118 is configured to receive input from the subject 101 and/or another user, and is further configured to provide visual output to the subject 101 and/or another user. In the illustrated embodiment of FIG. 1, the user interface 118 comprises a touchscreen display. In some embodiments, the user interface 118 may include, for example, one or more keypads, touchpads, touchscreens, trackballs, mice, and/or additional user interface devices or systems (e.g., a voice input/output system). Moreover, in some embodiments, one or more additional speakers 125 and/or microphones 126 separate from the device 110 may optionally be positioned near the subject 101, and communicatively coupled to the device 110 via the communication link 113 and/or another communication link. In some other embodiments, the device 110 may include one or more additional speakers and/or microphones (not shown).

In the illustrated embodiment of FIG. 1, the device 110 is depicted as a mobile phone (e.g., a smartphone). In other embodiments, however, the device 110 may comprise any suitable electronic device such as, for example, a tablet, a personal display assistant, a laptop computer, a desktop computer, a set top box and/or another electronic device configured to transmit and receive sound. In certain embodiments, the device 110 may comprise a component of one or more systems and/or devices (e.g., a baby monitor, a security system, an automobile entertainment system, a stereo system, a home intercom system, a clock radio). Moreover, in the illustrated embodiment of FIG. 1, the subject 101 (e.g., a human adult or a human child) is shown lying on a bed 104 (e.g., a bed in the subject's bedroom, a bed in a medical facility, a bed in a self-injection facility, etc.). In other embodiments, however, the subject 101 may be upright. In some embodiments, the system 100 may be configured to emit the sound 105 toward and receive the reflected sound 106 from one or more additional subjects (not shown).

In operation, the device 110 generates audio signals-including, for example, frequency modulated continuous wave (FMCW) audio signals—that sweep from a first frequency (e.g., about 18 kHz) to a second frequency (e.g., about 22 kHz). The first transducer 115 transmits the generated audio signals as the sound 105 toward the subject 101. A portion of the sound 105 is reflected and/or backscattered by the subject's chest or abdomen 103 toward the second transducer 116 as reflected sound 106. The second transducer 116 receives the reflected sound 106 and converts it into one or more electrical audio signals. As discussed in further detail below, the system 100 and/or the device 110 can be configured to detect peaks in the electrical audio signals that correspond to movements of the subject's chest or abdomen 103. The system 100 and/or the device 110 can be further configured to identify and/or disambiguate one or more events (e.g., respiratory depression, apnea, lack of gross motor movements, etc.) indicative of a potentially fatal opioid overdose absent intervention based on the detected peaks.

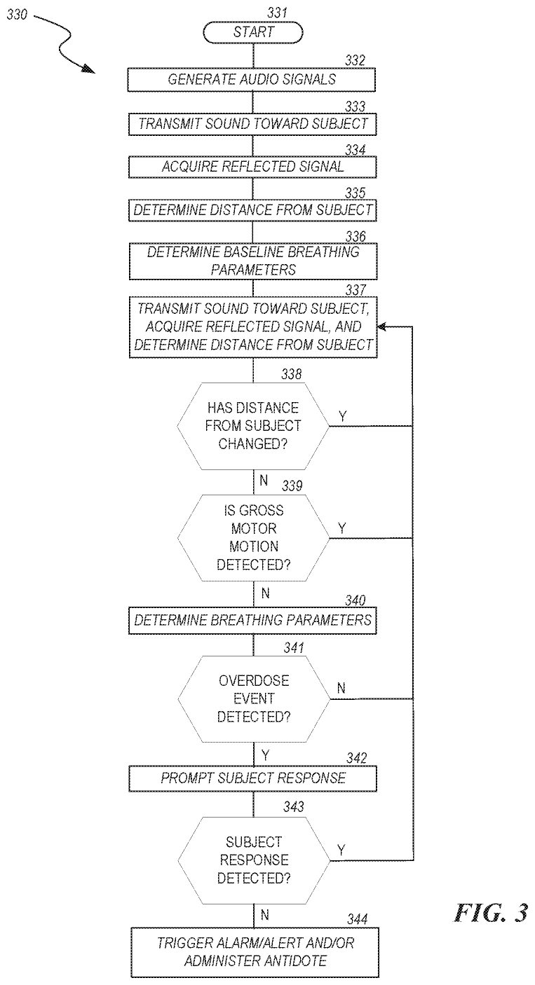

In some embodiments, the system 100 includes an antidote device or automatic release patch 129. The antidote device or patch 129 is configured to release an opioid antidote (e.g., naloxone or another opioid antidote) into the subject's body 102. The antidote device or patch 129 can be worn and/or connected to the subject 101 prior or shortly after opioids are introduced into the subject's body 102. As described in greater detail below with respect to FIG. 3, the system 100 can instruct the antidote device or patch 129 to release an antidote into the subject's body 102 when the system 100 identifies an event indicating a potentially fatal opioid overdose absent rescue intervention and/or when the subject is non-responsive.

The following discussion provides a brief, general description of a suitable environment in which the technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth), etc.). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s), a sound wave) over a period of time, or may be provided on any analog or digital network (packet switched, circuit switched, or other scheme).

FIG. 2 is a block diagram of an opioid overdose detection device and/or system 210 (e.g., the device 210 and/or the system 100 of FIG. 1) configured in accordance with embodiments of the present technology. The system 210 comprises several components including memory 211 (e.g., one or more computer readable storage modules, components, devices). In some embodiments, the memory 211 comprises one or more applications installed and/or operating on a computer and/or a mobile device (e.g., the device 110 of FIG. 1, a tablet, a smartphone, a PDA, a portable media player, or other “off-the-shelf” mobile device). The memory 211 can also be configured to store information (e.g., audio data, subject information or profiles, environmental data, data collected from one or more sensors, media files). A processor 212 (e.g., one or more processors or distributed processing elements) is coupled to the memory 211 and configured to execute operations and/or instructions stored thereon.

A speaker 215 (e.g., the first transducer 115 and/or the speaker 125 of FIG. 1) operatively coupled to the processor is configured to receive audio signals from the processor 212 and/or one or more other components of the system 210 and output the audio signals as sound (e.g., the sound 105 of FIG. 1). In some embodiments, the speaker 215 includes a conventional dynamic loudspeaker disposed in a mobile device (e.g., a smartphone or tablet). In some embodiments, the speaker 215 includes an earphone transducer and/or a standalone loudspeaker. In other embodiments, the speaker 215 includes a suitable transducer configured to output acoustic energy in at least a portion of the human audible frequency spectrum (e.g., between about 20 Hz and 22 kHz).

A microphone 216 (e.g., the second transducer 116 and/or the microphone 126 of FIG. 1) operatively coupled to the processor is configured to receive sound, convert the sound into one or more electrical audio signals and transmit the electrical audio signals to the memory 211 and/or the processor 212. In some embodiments, the microphone 216 includes a microphone disposed in a mobile device (e.g., a smartphone or tablet). In some embodiments, the microphone 216 is located on an earphone and/or along a cable connected to one or more earphones. In other embodiments, the microphone 216 includes another suitable transducer configured to receive acoustic energy in at least a portion of the human audible spectrum. Moreover, in some embodiments, the speaker 215 and the microphone 216 are spaced apart by a distance (e.g., 2 cm or greater, between about 2 cm and 10 cm, between 4 cm and 8 cm, or at least about 6 cm). In other embodiments, however, the speaker 215 is immediately adjacent the microphone 216. In certain embodiments, a single transducer can transmit sound energy and receive sound energy. In further embodiments, the speaker 215 and/or the microphone 216 comprise one or more additional transducers to form one or more transducer array(s). The transducer array(s) can be configured to transmit and/or receive beamformed audio signals.

Communication components 213 (e.g., a wired communication link and/or a wireless communication link (e.g., Bluetooth, Wi-Fi, infrared and/or another wireless radio transmission network)) communicatively couple the system 210 to one or more communications networks (e.g., a telecommunications network, the Internet, a WiFi network, a local area network, a wide area network, a Bluetooth network). A database 214 is configured to store data (e.g., audio signals and data acquired from a subject, equations, filters) used in the identification of movements of a subject. One or more sensors 217 are configured to provide additional data for use in motion detection and/or identification. The one or more sensors 217 may include, for example, one or more ECG sensors, blood pressure monitors, galvanometers, accelerometers, thermometers, hygrometers, blood pressure sensors, altimeters, gyroscopes, magnetometers, proximity sensors, barometers and/or hall effect sensors.

One or more displays 218 (e.g., the user interface 118 of FIG. 1) provide video output and/or graphical representations of data acquired and processed by the system 210. A power supply 219a (e.g., a power cable connected to a building power system, one or more batteries and/or capacitors) provides electrical power to components of the system 210. In embodiments that include one or more batteries, the power supply 219a can be configured to recharge, for example, via a power cable, inductive charging, resonant charging, and/or another suitable recharging method. Furthermore, in some embodiments, the system 210 optionally includes one or more other components 219b (e.g., one or more microphones, cameras. Global Positioning System (GPS) sensors, Near Field Communication (NFC) sensors).

As explained in further detail below, the system 210 is configured to transmit sound toward a subject and receive sound reflected off of the subject. The transmitted and received sound can be used by the system 210 to detect movement of the subject and identify one or more events (e.g., respiratory depression events, central apnea events, etc.) in the subject indicative of a potentially fatal opioid overdose absent intervention. In some embodiments, for example, the memory 211 includes instructions for generating audio signals (e.g., FMCW audio signals that sweep from about 18 kHz to about 22 kHz or higher) and providing the generated audio signals to the speaker 215. The speaker 215 transmits the audio signals as sound (e.g., acoustic energy comprising one or more waveforms) and directs at least a portion of the transmitted sound toward a subject (e.g., the subject 101 of FIG. 1) proximate the speaker 215. A portion of the sound is reflected or backscattered toward the microphone 216, which converts the sound into electrical audio signals. The memory 211 can further include instructions for processing the electrical audio signals to detect motion of the subject (e.g., movement of the subject's chest and/or abdomen), to disambiguate between periodic motion (e.g., respiratory motion) and non-periodic motion, and to identify one or more events (e.g., respiratory depression events, central apnea events, etc.) in the subject indicative of a potentially fatal opioid overdose absent intervention based on the detected motion of the subject. In some embodiments, an indication of the identified event can be output to the display 218 and/or can be transmitted via the communication component 213 to a medical professional (e.g., a nurse, a doctor, an EMT, a paramedic). In certain embodiments, the system 210 can be configured to determine baseline breathing information (e.g., breathing frequency) about a subject and store the baseline breathing information. The baseline breathing information can be compared to subsequent breathing measurements to identify a respiratory event.

FIG. 3 is a flow diagram illustrating a routine 330 for operating an opioid overdose detection system configured in accordance with various embodiments of the present technology. The routine 330 is executed, at least in part, by various components of an opioid overdose detection system. For example, all or a subset of one or more of the steps of the routine 330 can be carried out by a transducer (e.g., a speaker, a microphone, etc.), a communications link, and/or one or more other components of the system. Furthermore, the routine 330 can comprise a set of instructions stored on memory (e.g., the memory 211 of FIG. 2) and executed by one or more processors (e.g., the processor 212 of FIG. 2). In some embodiments, the routine 330 comprises one or more applications stored on a device (e.g., the device 110 of FIG. 1) of a system (e.g., the system 100 of FIG. 1).

The routine 330 begins at block 331 after the transducers are positioned proximate a subject (e.g., 1 m away from the subject, between about 0.5 m and 10 m from the subject, between about 1 m and 5 m from the subject). In some embodiments, the routine 330 can detect an orientation of the transducers in relation to the subject and, based on this detection, prompt a user to take corrective action. For example, the routine 330 may provide more accurate detection if a predetermined side of a measurement device (e.g., a front facing portion of the device 110 shown in FIG. 1) including the transducers is oriented at a predetermined orientation relative to the subject. In some embodiments, for example, it may be preferable to have a side of the measurement device on which a transducer (e.g., a speaker) is located or is most closely positioned to be oriented toward the subject. In embodiments in which the transducer and another transducer (e.g., a microphone) are not on the same side of the measurement device, however, it may be desirable to acquire audio from the subject if a side of the measurement device on which the other transducer is positioned is facing upright and/or substantially oriented toward the subject.

The routine 330 can be configured to determine an orientation of the measurement device using, for example, one or more sensing mechanisms (e.g., one or more gyroscopes, accelerometers, compass sensors, cameras). In some embodiments, for example, the one or more sensing mechanisms include one or more of the sensors 217 discussed above with reference to FIG. 2. In some embodiments, the routine 330 can generate one or more audible and/or visible indications instructing the subject and/or another user to take a corrective action based on the determined orientation. The corrective actions may include, for example, moving and/or orienting the measurement device toward the location of the subject. In some embodiments, the routine 330 may not proceed until one or more corrective actions are detected. Alternatively, the one or more audible and/or visible indications may persist while other blocks are executed in routine 330. In some embodiments, the routine 330 can be configured to adjust detection thresholds based on a detected orientation.

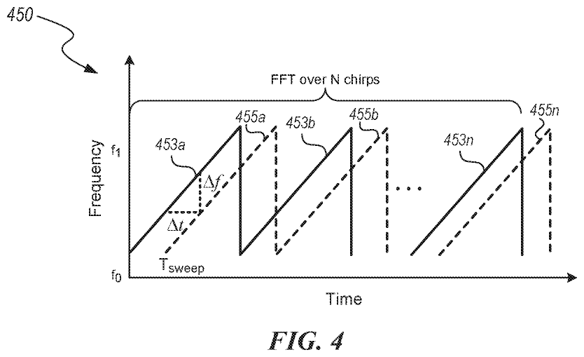

At block 332, the routine 330 generates one or more audio signals. In some embodiments, the audio signals include FMCW signals having a sawtooth waveform (see FIG. 4) that include a plurality of sweep audio signals or “chirps” that linearly sweep from a first frequency to a second, higher frequency. In some embodiments, the chirps sweep from a first frequency (e.g., about 18 kHz) to a second frequency (e.g., 22 kHz or higher). As those of ordinary skill in the art will appreciate, the frequency spectrum of a typical human ear ranges from 20 Hz to about 20 kHz, and many transducers are configured for playback over this spectrum. As humans age, however, the sensitivity of the ears to higher frequencies typically diminishes such that sounds having frequencies greater than about 18 kHz are effectively inaudible for a typical adult human. Accordingly, selecting the first and second frequencies to have a frequency equal to or greater than about 18 kHz allows for transmission of sound over a conventional loudspeaker configured for playback over a frequency range that will go unnoticed by and/or will not disturb most subjects. In other embodiments, the chirps sweep from a first frequency (e.g., 18 kHz) to a second frequency (e.g., a frequency greater than about 20 kHz and less than about 48 kHz, a frequency between about 22 kHz and about 44 kHz). In further embodiments, the chirps sweep between two frequencies outside the human audible range (e.g., greater than about 20 kHz and less than about 48 kHz). Moreover, in some embodiments, the routine 330 generates audio signals comprising FMCW signals having a sine waveform, a triangle waveform and/or a square waveform. In other embodiments, the routine 330 generates audio signals comprising pulse-modulated waveforms. In some embodiments, the routine 330 generates audio signals using another suitable modulation method.

At block 333, the routine 330 provides the generated audio signals to a speaker (e.g., the first transducer 115 of FIG. 1 and/or the speaker 215 of FIG. 2) configured to convert the audio signals to acoustic energy (e.g., the sound 105 of FIG. 1), and the routine 330 (e.g., continuously, periodically, sporadically) emits at least a portion of the acoustic energy toward the subject. At block 334, the routine 330 acquires a reflected signal using a microphone (e.g., the second transducer 116 of FIG. 1 and/or the microphone 216 of FIG. 2). The reflected signal includes data corresponding to a portion of the sound transmitted toward the subject at block 333, reflected or backscattered toward the microphone, and converted by the microphone to electrical signals.

FIG. 4 is a graph 450 depicting a reflected signal acquisition approach the routine 330 performs at blocks 332-334 in accordance with various embodiments of the present technology. Referring to FIGS. 3 and 4 together, the graph 450 includes a plurality of transmitted signals 453 (identified individually as a first transmitted signal 453a, a second transmitted signal 453b, and an nth transmitted signal 453n) that are generated by the routine 330 at block 332 and emitted by the routine 330 at block 333. The graph 450 also includes a plurality of corresponding reflected signals 455 (identified individually as a first reflected signal 455a, a second reflected signal 455b, and an nth reflected signal 455n) that are acquired by the routine 330 at block 333. The plurality of transmitted signals 453 comprise FMCW signals that linearly sweep between a first frequency f₀ (e.g., 18 kHz) and a second, higher frequency f₁ (e.g., 22 kHz or higher) over a time T_{sweep} (e.g., between about 5 ms and about 15 ms, between about 9 ms and about 11 ms, or about 10 ms). The chirp duration, T_{sweep}, is selected so that the reflections from all points within an operational distance (e.g., the distance D of FIG. 1) preferably start arriving before the chirp ends. In one particular embodiment, for example, the operational distance is approximately 1 meter, and a chirp duration T_{sweep} of 10 ms is selected, which provides a frequency resolution of 100 Hz.

The individual transmitted signals 453 are emitted from a speaker (e.g., the first transducer 115 of FIG. 1) and a corresponding one of the reflected signals 455 is received at a microphone (e.g., the second transducer 116 of FIG. 2) a period of time after the transmitted signals 453 are emitted from the loudspeaker. For example, the first transmitted signal 453a is emitted from the speaker toward a subject and the corresponding first reflected signal 455a is received by the microphone a time delay Δt later. The time delay Δt is given by:

$$Δ t = \frac{2 d}{Vsound} \qquad ( 1 )$$

in which d is the distance between the loudspeaker and the subject and V_{sound} is the velocity of sound (e.g., approximately 340 m/s at sea level). Since the transmitted frequency increases linearly in time, time delays in the reflected signals translate to frequency shifts in comparison to the transmitted signals. The frequency shift Δf between individual transmitted signals and the corresponding reflected signals is given by the following:

$$Δ f = \frac{{f}_{1} - {f}_{0}}{{T}_{sweep}} Δ t \qquad ( 2 )$$

With multiple reflectors at different distances from the receiver, their reflections translate to different frequency shifts in the signal. In this manner, the distance between a portion of the subject's body (e.g., the subject's arms, legs, head, chest, abdomen, etc.) and the measurement device is captured as frequency shifts in the reflected signal acquired at block 334. By monitoring changes in the distance between the portion of the patient's body and the measurement device, motion (e.g., gross motor motion, breathing motion, etc.) can be monitored.

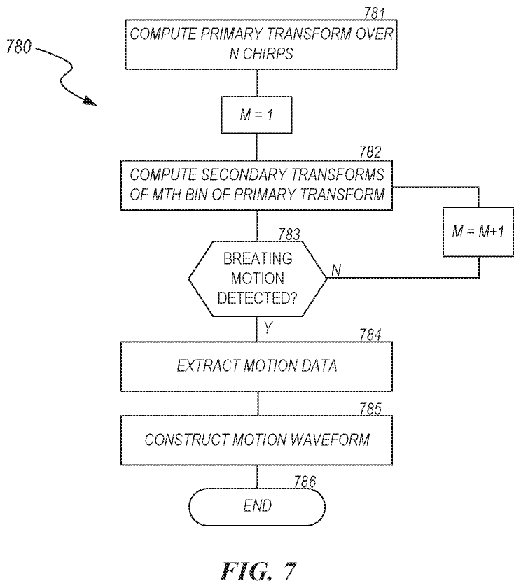

Referring again to FIG. 3, the routine 330 at blocks 335 and 336 analyzes the transmitted and reflected audio signals to detect frequency shifts and extract motion data of the subject. At block 335, the routine 330 uses the signals to determine a distance between the measurement device and the subject. In some embodiments, as described in greater detail below with respect to FIG. 7, the routine 330 computes a fast Fourier transform (FFT) of the reflected signal. For example, the routine 330 computes an FFT over an integer number of chirp durations (as shown in FIG. 4). Computing an FFT over N chirps decreases a width of each resulting FFT frequency bin by a factor of N. In one embodiment, an FFT computed over 15 chirps with a sweep duration T_{sweep} of 10 ms results in a frequency resolution of 6.66 Hz, allowing the capture of depressed breathing motion down to a chest movement of 0.7 cm. Although taking an FFT over several chirps averages (and hence loses) any high frequency motion, the average breathing rate of human subjects is less than 20 breaths per minute (which is relatively low frequency motion). Thus, no significant breathing motion is lost in this procedure.

After computing the FFT of the reflected signal, the routine 330 analyzes the resulting frequency bins for motion data of the subject. Each frequency bin corresponds to a distance away from the measurement device. Thus, a motion signal of the subject will be present in a unique frequency bin corresponding to the distance between the subject and the measurement device. In other words, identifying motion data of the subject in a frequency bin of the FFT provides an indication that the subject is a distance corresponding to the frequency bin away from the measurement device.

In some embodiments, the FFT analyzes the resulting frequency bins of the FFT starting with the first frequency bin (corresponding to zero meters away from the measurement device) and proceeds to the next frequency bin until motion data is identified. In other embodiments, the routine 330 analyzes each frequency bin of the FFT regardless of whether the motion data is identified in an earlier frequency bin. In these embodiments, the routine 330 can identify multiple motion signals corresponding to multiple subjects that are differing distances away from the measurement device. In some embodiments, the routine 330 can monitor each of the identified motion signals in accordance with the discussion below. In other embodiments, the routine 330 can monitor a subset of the identified motion signals (e.g., the motion signal identified closest to the measurement device) in accordance with the discussion below. In some embodiments, the routine 330 can proceed to block 342 to prompt a response from the subject if the routine is unable to identify motion data in any of the frequency bins of the FFT.

At block 336, the routine 330 determines baseline breathing parameters of the subject using the motion data identified and extracted from the reflected signal at block 335. As discussed in greater detail below, the routine 330 can use baseline breathing parameters to detect events indicating a potentially fatal overdose absent intervention. For example, the routine 330 can use baseline breathing parameters as a reference for a subject. Thus, the routine 330 can determine that an abnormally low respiratory rate of a human, for example, is normal for a particular subject (or is a result of another instance of the subject using opioids early in the day). The routine 330 determines the breathing measurements of the subject by identifying and analyzing peaks in the motion waveform extracted at block 335.

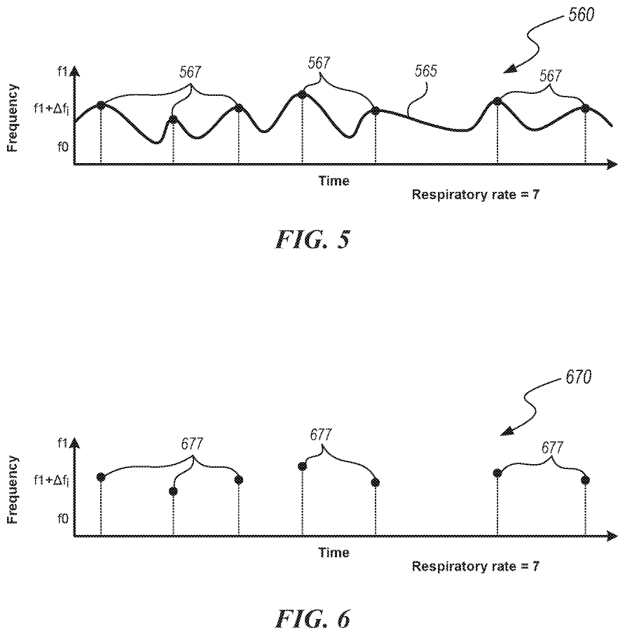

FIG. 5 is a graph 560 illustrating an example motion waveform 565 (e.g., a breathing signal) extracted from a frequency bin of an FFT of a reflected signal over N chip durations. As shown in FIG. 5, the motion waveform 565 illustrated in the graph 560 includes seven peaks 567. FIG. 6 is a graph 670 illustrating several peaks 677 that correspond to the peaks 567 the routine 330 identified in the motion waveform 565 of FIG. 5. Referring to FIGS. 3, 5, and 6 together, in some embodiments, the routine 330 determines the subject's respiratory rate by determining the number of peaks present in the motion waveform within a 60-second interval. For example, assuming the x-axis of FIG. 5 includes a 30 second interval, the routine 330 determines the subject's respiratory rate is 14 breaths per minute (7 peaks/30 sec=14 peaks/60 sec). A respiratory rate (e.g., a single respiratory rate, an average respiratory rate, a sliding respiratory rate) calculated before or shortly after opioids are introduced into the subject's body is used as a baseline respiratory rate for the subject. In these and other embodiments, the routine 330 determines the amount of time separating individual peaks in the motion waveform. In some embodiments, the routine 330 uses an average amount of time separating successive (e.g., immediately adjacent) peaks in the motion waveform before or shortly after opioids are introduced into the subject's body as a baseline. In these and still other embodiments, the routine 330 determines a peak amplitude and/or a peak prominence of an identified peak. In some embodiments, the routine 330 uses an average peak amplitude and/or an average peak prominence calculated before or shortly after opioids are introduced into the subject's body as baseline parameters or measurements. In some embodiments, the routine 330 leverages the periodicity of the breathing signal. For example, the routine 330 can use only peaks that are separated by a minimum specified number of samples (e.g., 20 samples corresponding to a maximum breathing rate of 20 breaths per minute). In these and other embodiments, the routine 330 updates one or more of the breathing parameters (e.g., the baseline or other breathing parameters) by taking a respective weighted average of the one or more breathing parameters over consecutive periods.

Breathing displacement of the chest and abdomen is relatively small while opioids are acting on the central nervous system of the subject and therefore cause small frequency shifts in the reflected signal. For a FMCW signal with a 4 kHz bandwidth, breathing displacement while opioids are acting on the central nervous system of the subject, for example, may result in less than an 8.33 Hz frequency shift. Tus, small amplitude motion can introduce noise into the breathing signal and cause errors in breathing measurements. For this reason, the routine 330 in some embodiments filters the motion data extracted from the reflected signal before determining the breathing parameters. For example, the routine 330 feeds the motion data through a bandpass decimating Cascaded Integrated Comb filter to remove noise higher than a selected frequency (e.g., 1 Hz) and to decimate the signal by a ratio (e.g., a ratio of two).

Referring again to FIG. 3, at block 337, the routine 330 transmits sound toward the subject and acquires corresponding reflected signals in a manner similar to the routine 330 at blocks 333 and 334. Additionally, the routine 330 determines the distance between the subject and the measurement device in a manner similar to the routine at block 335 above. In some embodiments, in contrast to block 335, the routine 330 at block 337 first monitors the frequency bin corresponding to the last determined distance between the subject and the measurement device. If motion data is still present within that frequency bin, the routine 330 determines that the distance between the subject and the measurement device has not changed (block 338) and proceeds to block 339. On the other hand, if motion data is not present within the corresponding frequency bin, the routine 330 searches other frequency bins of the FFT (e.g., starting with the frequency bin corresponding to the smallest distance from the measurement device) for motion data, as discussed above with respect to block 335. If the routine 330 identifies motion data corresponding to the subject in another frequency bin, the routine 330 determines that the subject has significantly moved with respect to the measurement device. In some embodiments, the significant movement of the subject can be interpreted as an indication that the subject is not currently exhibiting signs of a potentially fatal opioid overdose. Thus, the routine 330 can return to block 337. In other embodiments, the routine 330 can proceed to block 339 to determine whether gross motor motion is detected in the motion data identified in a different frequency bin. In some embodiments, the routine 330 can proceed to block 342 to prompt a response from the subject if the routine 330 is unable to locate motion data in any of the frequency bins.

At block 339, the routine 330 determines whether gross motor motion of the subject is present in the motion data extracted from the reflected signal at block 337. As discussed above, breathing displacement of the chest and abdomen is relatively small while opioids are acting on the central nervous system of the subject and therefore cause small frequency shifts in the reflected signal. In contrast, gross motor motion (e.g., movements of the subject's hands, arms, feet, legs, head, etc.) causes relatively large frequency shifts in the reflected signal. Because portions of the subject's body that typically exhibit gross motor motion are close to the subject's chest and abdomen from which breathing motion is captured and are at approximately the same distance from the measurement device as the subject's chest and abdomen, the frequency shifts caused by gross motor motions can be added to the frequency shifts attributable to breathing motion in the same frequency bin. As gross motor motion has higher amplitudes compared to amplitudes of more subdued breathing motion (and therefore often overpowers the breathing motion data), gross motor motion can make it difficult to extract a breathing motion signal from the reflected signal. Nevertheless, the presence of gross motor motion indicates that the subject is active and thus not overdosed. Therefore, the routine 330 differentiates breathing signals that have periodic, low-frequency, and low-amplitude motion from gross motor motion signals that have aperiodic, high-frequency, and high-amplitude motion. In some embodiments, the routine 330 identifies gross motor motion by analyzing the peaks in an extracted motion waveform in a manner similar to the routine 330 at block 336. For example, the routine 330 analyzes the frequency and amplitudes of the peaks present in the motion waveform. If the routine 330 determines that the motion waveform includes peaks having higher frequencies and larger amplitudes (e.g., two times larger) than that of typical breathing peaks, the routine 330 determines that gross motor motion is present in the motion waveform and that the subject is not currently exhibiting signs of a potentially fatal opioid overdose. In this case, the routine 330 returns to block 337 and continues to monitor the subject. Otherwise, if the routine 330 determines that gross motor motion is not detected in the motion waveform (or is present for only a few seconds), the routine 330 determines that the motion waveform includes breathing motion data and proceeds to block 340.

At block 340, the routine 330 determines one or more breathing parameters of the subject using peaks in the motion waveform extracted from the reflected signal. In some embodiments, the routine 330 uses the baseline breathing parameters calculated at block 336 before or shortly after opioids are introduced into the subject's body to identify peaks in the motion waveform. In some embodiments, the routine 330 determines the breathing parameters in a manner similar to the routine 330 at block 336.

At block 341, the routine 330 identifies events indicating a potentially fatal opioid overdose absent intervention. For example, the routine 330 determines whether a respiratory depression event or a central apnea event is detected in the motion waveform, both of which indicate or precede a fatal opioid overdose. In some embodiments, the routine 330 identifies the events by comparing the breathing parameters determined at block 340 to threshold breathing parameters. As an example, the routine 330 compares a respiratory rate calculated at block 340 to a threshold respiratory rate (e.g., seven breaths per minute). If the respiratory rate calculated at block 340 is equal to or less than the threshold respiratory rate, the routine 330 determines that the subject is experiencing a respiratory depression event and proceeds to block 342. As another example, the routine 330 compares the amount of time elapsed between two successive peaks identified in the motion waveform at block 340 to a threshold amount of time (e.g., 10 seconds). If the amount of time between the two successive peaks is equal to or greater than the threshold amount of time, the routine 330 determines that the subject is experiencing a central apnea event and proceeds to block 342. In some embodiments, the routine 330 compares the breathing parameters calculated at block 340 to the baseline breathing parameters calculated at block 336 to identify opioid overdose events. For example, the routine 330 can determine that the subject is experiencing an opioid overdose event if one or more of the breathing parameters calculated at block 340 differ from one or more respective baseline breathing parameters by equal to or greater than a threshold difference and/or by equal to or greater than a threshold difference over a specified period of time. If the routine 330 does not detect an event indicating a potentially fatal opioid overdose absent intervention, the routine 330 returns to block 337.

At block 342, the routine 330 prompts the subject for a response. In some embodiments, the routine 330 prompts the subject for a response by triggering an audio and/or visual alert and/or alarm. At block 343, the routine 330 determines whether the subject has responded. In some embodiments, the routine 330 can determine whether the subject has responded to the alert/alarm by determining whether the subject has interacted with the system (e.g., pushed a button), by determining whether the subject has exhibited large gross motor movement since the alert/alarm was triggered, and/or by determining whether the subject has responded in another manner. In some embodiments, the routine 330 can escalate the alert/alarm over time (e.g., by making the alert/alarm louder, by altering displayed colors, by flashing an alert, etc.) until the subject responds. In these and other embodiments, the routine 330 can continue to monitor the subject (e.g., by repeating blocks 337-342) and/or can accelerate the escalation of the alert/alarm if the routine 330 detects that the subject's breathing parameters are deteriorating. If the routine 330 determines that the subject responded to the alert/alarm, the routine 330 can return to block 337. On the other hand, if the routine 330 determines that the subject has not responded to the alert/alarm (e.g., within a specified period of time) and/or if the routine 330 determines that the subject's breathing parameters indicate a large risk of fatal opioid overdose absent intervention, the routine 330 can proceed to block 344.

At block 344, the routine 330 solicits rescue intervention and/or administers an opioid antidote. In some embodiments, the routine 330 solicits rescue intervention by initiating calls or alerts to emergency services (e.g., by dialing 911 and/or by sending the subject's current location to EMT's or paramedics). In these and other embodiments, the routine 330 solicits rescue intervention by initiating calls or alerts to family members or friends (e.g., emergency contacts specified by the subject). In these and other embodiments having an antidote device or an automatic release patch 129 that is currently worn by and/or connected to the subject, the routine 330 can instruct the antidote device or patch to release an opioid antidote (e.g., naloxone or another opioid antidote) into the subject's body 102.

Although the steps of the routine 330 are discussed and illustrated in a particular order, the method illustrated by the routine 330 in FIG. 3 is not so limited. In other embodiments, the method can be performed in a different order. For example, any of the steps of the routine 330 can be performed before, during, and/or after any of the other steps of the routine 330. Moreover, a person of ordinary skill in the relevant art will readily recognize that the illustrated method can be altered and still remain within some embodiments of the present technology. For example, one or more steps of the routine 330 illustrated in FIG. 3 can be omitted and/or repeated in some embodiments.

In some embodiments, the routine 330 can record and/or output data collected and/or analyzed during the routine 330 illustrated in FIG. 3. For example, the routine 330 may record all or a portion of data relating to motion waveforms identified in acquired reflected signals, distance determinations between the subject and the measurement device, gross motor motion events, calculated breathing parameters, identified events indicating potentially fatal opioid overdoses absent intervention, triggered alerts/alarms, detected subject responses (or lack thereof), rescue intervention solicitations, and/or administration of opioid antidotes. In some embodiments, the routine 330 stores the records in a memory or database (e.g., the memory 211 and/or the database 214 of FIG. 2). In these and other embodiments, the routine 330 can output one or more of the records in a report and/or to a display (e.g., the user interface 118 of FIG. 1 and/or the display 218 of FIG. 2).

FIG. 7 is a flow diagram of a routine 780 for detecting motion data in and constructing a motion waveform from a reflected audio signal in accordance with various embodiments of the present technology. The routine 780 is executed, at least in part, by various components of an opioid overdose detection system. For example, all or a subset of one or more of the steps of the routine 780 can be carried out by a transducer (e.g., a speaker, a microphone, etc.), a communications link, an FMCW receiver, and/or one or more other components of the system. Furthermore, the routine 780 can comprise a set of instructions stored on memory (e.g., the memory 211 of FIG. 2) and executed by one or more processors (e.g., the processor 212 of FIG. 2). In some embodiments, the routine 780 comprises one or more applications stored on a device (e.g., the device 110 of FIG. 1) of a system (e.g., the system 100 of FIG. 1).

The routine 780 begins at block 781 with monitoring a plurality of transmit/receive cycles as described above in reference to blocks 332-334 of FIG. 3 and to FIG. 4. The routine 780 receives a plurality of reflected signals (e.g., the reflected signals 455 of FIG. 4) and computes a plurality of primary frequency transforms (e.g., FFT's) over a predetermined number N (e.g., 5, 10, 15, 20, 40, 50) of chirps or transmit/receive cycles. As those of ordinary skill in the art will appreciate, a frequency transform converts and/or demodulates a signal from a first domain (e.g., a time domain) to a frequency domain. The primary transforms computed by the routine 780 at block 781 represent frequency spectra of the reflected signals in a plurality of frequency bins. Each bin represents a discrete portion of the frequency spectrum of the reflected signals. In some embodiments, for example, the routine 780 computes a plurality of 5120-point FFTs over every series of 15 reflected signals received by the routine 780.

At block 782, the routine 780 computes a secondary frequency transform (e.g., an FFT) of an individual bin of each of the primary transforms computed at block 781 over a predetermined time duration (e.g., 5 s, 10 s, 30 s, 60 s, 5 minutes, 10 minutes). When the routine 780 initially proceeds to block 782, an index value m is set to 1. Accordingly, the routine 780 performs an FFT of the 1^{st} bin of a plurality of the primary transforms as a function of time. The 1^{st} bin corresponds to a distance of zero meters from the measurement device. In some embodiments, for example, the routine 780 computes a 24,000-point FFT of the 1^{st} bin of a plurality of primary transforms over a time duration of 30 seconds.

At block 783, the routine 780 analyzes the secondary transform calculated at block 782 to determine whether the second transform includes one or more peaks associated with breathing frequencies. In some embodiments, for example, the routine 780 analyzes the secondary transform from block 782 to determine if any peaks are detected between about 0.1 Hz or about 0.9 Hz (e.g., between about 0.5 Hz and about 0.7 Hz), which is a range that includes typical human breathing frequencies. If no peaks are detected at or near these frequency values, then the routine 780 returns to block 782 and adds 1 to the index value m (i.e., m+1). The routine 780 computes a new secondary transform at block 782 at the next bin m of the primary transforms over a predetermined period of time. The routine 780 continues to iteratively compute secondary transforms until the routine 780 detects peaks corresponding to breathing frequencies and/or until a predetermined value of m (e.g., 58, 60, 100, 200) is reached. If the routine 780 detects a peak between about 0.1 Hz and about 0.9 Hz, the routine 780 stores the index m corresponding to the bin number in which the peak is detected as mₚₑₐₖ, and proceeds to block 784. In some embodiments, in the worst-case scenario, the routine 780 iterates through 48 bins before isolating a breathing signal.

At block 784, the routine 780 extracts motion data from the reflected audio signals. In some embodiments, the routine 780 continues to compute a plurality of the primary transforms of the reflected audio and compute a secondary transform of bin mₚₑₐₖ of the primary transforms as a function of time. The routine 780 can also compute a distance D between a measurement device (e.g., the device 110 of FIG. 1) and the subject using the mₚₑₐₖ index obtained by the routine 780 at block 783. For example, if the bandwidth of each bin is b_{width} (Hz) and breathing motion detected is detected in the mₚₑₐₖ^{th} bin of the primary transform of block 781), the resulting frequency shift caused by movement of the subject is approximately b_{width}*mₚₑₐₖ*2). Using equation 1 above, the time delay and the corresponding distance from the measurement device can be obtained.

At block 785, the routine 780 constructs a motion waveform (e.g. the motion waveform 565 of FIG. 5) of movement of the subject's chest and/or abdomen as a function of time using the secondary transform computed at block 782. In some embodiments, in accordance with the discussion above with respect to FIG. 3, the motion waveform can be analyzed to calculate breathing parameters, detect gross motor motion, and/or identify events indicating a potentially fatal opioid overdose absent intervention.

At block 786, the routine 780 ends. In some embodiments, the routine 780 returns to block 781 to compute a primary transform over the next N number of chirps. In some embodiments, the routine 780 resets the index value m before returning to block 781 such that the routine 780 computes a secondary transform at block 782 on the 1^{st} bin of the primary transform computed at block 781. In other embodiments, the routine 780 does not reset the index value m when returning to block 781 such that the routine 780 computes a secondary transform at block 782 on a frequency bin of the primary transform computed at block 781 in which motion data was identified in the previous N number of chirps. If the routine 780 determines that motion data is not present in the frequency bin previously containing motion data, the routine 780 can increment or decrement the index value m (e.g., by one), reset the index value m back to 1, and/or compute a secondary transform on another frequency bin.

Although the steps of the routine 780 are discussed and illustrated in a particular order, the method illustrated by the routine 780 in FIG. 7 is not so limited. In other embodiments, the method can be performed in a different order. For example, any of the steps of the routine 780 can be performed before, during, and/or after any of the other steps of the routine 780. Moreover, a person of ordinary skill in the relevant art will readily recognize that the illustrated method can be altered and still remain within some embodiments of the present technology. For example, one or more steps of the routine 780 illustrated in FIG. 7 can be omitted and/or repeated in some embodiments.

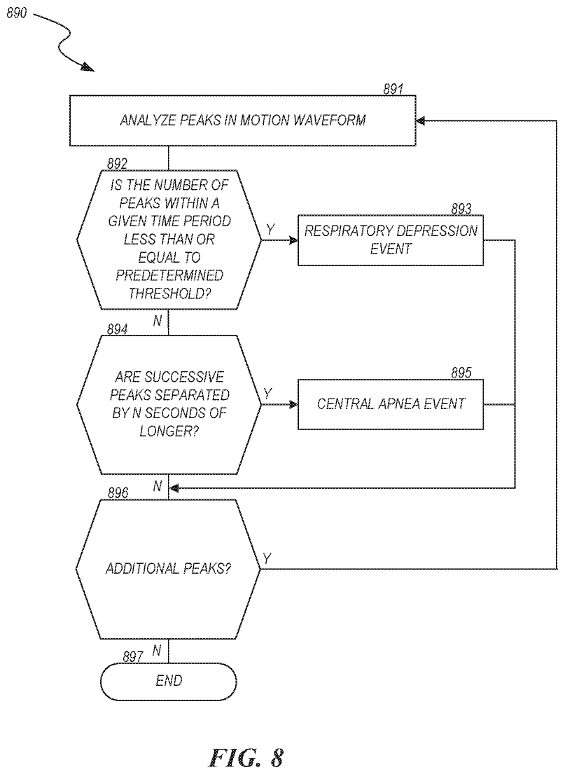

FIG. 8 is a flow diagram of a routine 890 for identifying events indicating a potentially fatal opioid overdose absent intervention in accordance with various embodiments of the present technology. The routine 890 is executed, at least in part, by various components of an opioid overdose detection system. For example, all or a subset of one or more of the steps of the routine 890 can be carried out by a transducer (e.g., a speaker, a microphone, etc.), a communications link, an FMCW receiver, and/or one or more other components of the system. Furthermore, the routine 890 can comprise a set of instructions stored on memory (e.g., the memory 211 of FIG. 2) and executed by one or more processors (e.g., the processor 212 of FIG. 2). In some embodiments, the routine 890 comprises one or more applications stored on a device (e.g., the device 110 of FIG. 1) of a system (e.g., the system 100 of FIG. 1).

At block 891, the routine 890 analyzes peaks in a motion waveform (e.g., the peaks 567 identified in the motion waveform 565 of FIG. 5). In some embodiments, the routine 890 uses baseline breathing parameters of a subject to identify peaks in the motion waveform corresponding to the subject's breathing. In these and other embodiments, the 890 determines one or more breathing parameters based on the identified peaks. For example, the routine 890 determines the subject's respiratory rate by determining the number of peaks identified in the motion waveform within a 60-second interval. In these and other embodiments, the routine 890 determines the amount of time separating successive peaks identified in the motion waveform. In these and still other embodiments, the routine 890 determines a peak amplitude and/or a peak prominence of an identified peak. In some embodiments, the routine 890 leverages the periodicity of the breathing signal. For example, the routine 890 only analyzes/uses peaks that are separated by a minimum specified number of samples (e.g., 20 samples corresponding to a maximum breathing rate of 20 breaths per minute).

At block 892, the routine 890 determines whether the number of peaks identified within a given period of time is less than or equal to a predetermined threshold number of peaks per 60 second interval. In some embodiments, the predetermined threshold number of peaks per 60 second interval is set at seven peaks or less such that identification of seven peaks or less in a 60 second interval is identified as a respiratory depression event. A seven peak per minute threshold corresponds to the rate at which the Agency for Healthcare Research and Quality (AHRQ) recommends employing a hospital's Rapid Response System. If the routine 890 identifies seven peaks or less in the motion waveform within a given 60-second interval, the routine 890 identifies a respiratory depression event at block 893. Otherwise, the routine 890 proceeds to block 896.

At block 894, the routine 890 determines whether successive peaks identified in the motion waveform are separated by a time duration greater than a predetermined threshold duration of time (e.g., 10 seconds). In some embodiments, the predetermined threshold duration of time is set at 10 seconds or greater such that the absence of breathing for 10 seconds or more is identified as a central apnea event. A 10-second predetermined threshold duration of time corresponds with the Food and Drug Administration (FDA) definition of an apnea event and the requirement for FDA-approved devices to detect this threshold. If the routine 890 detects the successive peaks identified in the motion waveform are separated by a duration of time equal to or greater than the predetermined threshold duration of time, the routine 890 identifies a central apnea event at block 895. Otherwise, the routine 890 proceeds to block 896.

In some embodiments, the routine 890 can use one or more other breathing parameters than discussed above with respect to block 892-895 to identify events indicating a potentially fatal opioid overdose absent intervention. For example, the routine 890 can use peak amplitude and/or peak prominence. The one or more other breathing parameters can be compared to baseline breathing parameters or one or more other, previously-determined breathing measurements. In these embodiments, the routine 890 can identify opioid overdose events if a change in a breathing parameter exceeds a predetermined threshold change and/or a predetermined threshold change over time.

At decision block 896, the routine 890 determines whether there are additional peaks in the motion waveform. If there are additional peaks in the motion waveform, the routine 890 returns to block 891. Otherwise, the routine 890 ends at block 897.

Although the steps of the routine 890 are discussed and illustrated in a particular order, the method illustrated by the routine 890 in FIG. 8 is not so limited. In other embodiments, the method can be performed in a different order. For example, any of the steps of the routine 890 can be performed before, during, and/or after any of the other steps of the routine 890. Moreover, a person of ordinary skill in the relevant art will readily recognize that the illustrated method can be altered and still remain within some embodiments of the present technology. For example, one or more steps of the routine 890 illustrated in FIG. 8 can be omitted and/or repeated in some embodiments.

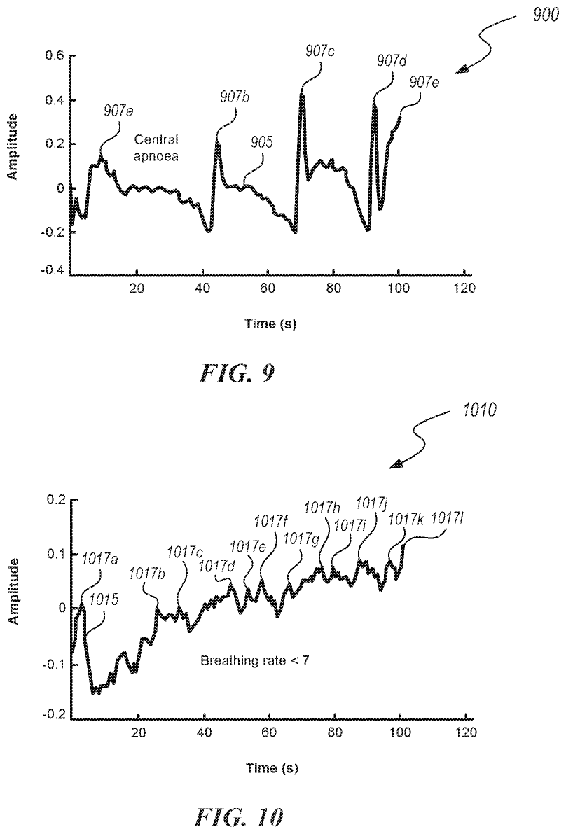

FIGS. 9 and 10 show examples of events indicating potentially fatal opioid overdoses absent intervention that may be identified by the routines 330 and/or 890 (FIGS. 3 and 8) in accordance with various embodiments of the present technology. FIG. 9, for example, is a graph 900 depicting one example of central apnea events described above with reference to block 341 of FIG. 3 and blocks 892 and 893 of FIG. 8. A breathing motion waveform 905 includes several pairs of successive peaks (907a and 907b, 907b and 907c, 907c and 907d) where the peaks of each pair are separated by an amount of time equal to or greater than a predetermined threshold amount of time (e.g., about 10 s). The several pairs of successive peaks illustrate that the corresponding subject underwent several central apnea events followed by a deep breath (shown at peak 907e).

FIG. 10 is a graph 1010 depicting one example of a respiratory depression event described above with reference to block 341 of FIG. 3 and to blocks 894 and 895 of FIG. 8. A motion waveform 1015 includes a plurality of peaks 1017 (identified individually as peaks 1017a-10171). As shown, the prominence of each peak is much less than the prominence of each peak in the motion waveform 565 of FIG. 5 or the motion waveform 905 of FIG. 9. Furthermore, the 11 peaks identified in the waveform 1015 correspond to 11 breaths that were taken over the span of approximately 100 seconds, which corresponds to a breathing rate of less than seven breaths per minute. Thus, the number of the peaks 1017 over a given amount of time (e.g., 100 seconds) illustrate the corresponding subject underwent a respiratory depression event.

### B. Examples

Several aspects of the present technology are set forth in the following examples.

1. A method of operating an electronic device for detecting events indicating respiratory failure, the method comprising:
transmitting acoustic energy toward a subject using a first transducer of the electronic device, wherein the transmitted acoustic energy increases from a first frequency to a second frequency, and wherein the second frequency is outside of the human audible spectrum of acoustic signals;
acquiring a reflected signal corresponding to the transmitted acoustic energy using a second transducer of the electronic device, wherein the second transducer is configured to produce electrical signals corresponding to the acquired reflected signal;
identifying motion data of the subject in the reflected signal; and determining whether the subject is experiencing an event indicating respiratory failure based, at least in part, on the identified motion data.

2. The method of example 1 wherein identifying the motion data further includes:
computing a primary transform of the reflected signal;
searching frequency bins of the primary transform for the motion data;
identifying a frequency bin containing the motion data; and determining a distance between the subject and the second transducer, wherein the distance corresponds to the identified frequency bin.

3. The method of example 2 wherein searching the frequency bins of the primary transform includes sequentially searching the frequency bins starting with a frequency bin corresponding to a shortest distance from the second transducer and until the motion data is identified in the identified frequency bin.

4. The method of example 2 wherein the motion data is second motion data, and wherein searching the frequency bins of the primary transform includes searching the frequency bins for the second motion data starting with a previously-identified frequency bin containing first motion data corresponding to a previously acquired portion of the reflected signal.

5. The method of example 4 wherein the identified frequency bin is different than the previously-identified frequency bin, and wherein determining whether the subject is experiencing an event indicating respiratory failure based, at least in part, on the identified second motion data includes determining the subject is not experiencing an event indicating respiratory failure.

6. The method of any one of examples 2-4 wherein:
the subject is a first subject, the identified motion data is first identified motion data, the identified frequency bin is a first identified frequency bin, and the distance is a first distance;
the method further comprises identifying second motion data corresponding to a second subject in a second frequency bin of the primary transform of the reflected signal; and
identifying the second motion data includes determining a second distance between the second subject and the second transducer, wherein the second distance corresponds to the second identified frequency bin.

7. The method of example 6, further comprising determining whether the second subject is experiencing an event indicating respiratory failure based, at least in part, on the second identified motion data.

8. The method of any one of examples 1-7 wherein determining whether the subject is experiencing an event indicating respiratory failure includes:
determining that gross motor motion of the subject is present in the motion data; and
determining that the subject is not experiencing an event indicating respiratory failure.

9. The method of any one of examples 1-8 wherein determining whether the subject is experiencing an event indicating respiratory failure includes:
determining one or more breathing parameters of the subject based at least in part on the identified motion data; and
comparing at least one of the one or more breathing parameters to at least one corresponding predetermined threshold.

10. The method of example 9 wherein determining whether the subject is experiencing an event indicating respiratory failure further includes filtering the motion data to remove frequency higher than 1 Hz.

11. The method of example 9 or example 10 wherein determining the one or more breathing parameters includes:
identifying peaks in the identified motion data; and
using at least one of the identified peaks to calculate the one or more breathing parameters, wherein the one or more breathing parameters include a respiratory rate of the subject, an amount of time separating successive peaks identified in the identified motion data, an amplitude of at least one identified peak, a prominence of at least one identified peak, or a combination thereof.

12. The method of example 11 wherein identifying peaks in the identified motion data includes using previously determined baseline breathing parameters of the subject to identify the peaks, considering only peaks separated by twenty samples of the motion data or more, or a combination thereof.

13. The method of example 11 or example 12 wherein:
the one or more breathing parameters include a respiratory rate of the subject, an amount of time separating successive peaks identified in the identified motion data, or a combination thereof;
comparing the one or more breathing parameters include comparing the respiratory rate to a threshold respiratory rate, comparing the amount of time to a threshold amount of time, or a combination thereof;
determining that the respiratory rate of the subject is equal to or less than the threshold respiratory rate, the amount of time separating successive peaks identified in the identified motion data is equal to or greater than the threshold amount of time, or a combination thereof; and
determining whether the subject is experiencing an event indicating respiratory failure further includes determining the subject is experiencing a respiratory depression event, a central apnea event, or a combination thereof.

14. The method of example 13, further comprising triggering an alert or alarm to prompt the subject for a response.

15. The method of example 14, further comprising:
determining the subject has not responded to the alert or alarm within a predetermined amount of time, determining the subject is deteriorating based at least in part on a breathing parameter of the subject determined after triggering the alert or alarm, or a combination thereof; and
escalating the alert or alarm by soliciting rescue intervention from emergency services, soliciting rescue intervention from a contact previously specified by the subject, or a combination thereof.

16. The method of example 14 or example 15, further comprising:
determining the subject has not responded to the alert or alarm within a predetermined amount of time, determining the subject is deteriorating based at least in part on a breathing parameter of the subject determined after triggering the alert or alarm, or a combination thereof; and
administering or instructing another device to administer an antidote to the subject.

17. A method of operating a mobile device to identify opioid overdose indicators, the method comprising:
transmitting sound toward a subject using a first transducer of the mobile device, wherein frequency of the transmitted sound increases from a first frequency to a second frequency over time, and wherein the second frequency is outside of the human audible spectrum of acoustic signals;
acquiring a reflected sound signal corresponding to the transmitted sound using a second transducer of the mobile device, wherein the second transducer is configured to produce electrical signals corresponding to the acquired reflected sound signal;
extracting motion data of the subject from the reflected sound signal;
determining a distance between the subject and the mobile device based at least in part on the reflected sound signal; and
determining whether the subject is currently in need of rescue intervention based, at least in part, on the extracted motion data.

18. The method of example 17 wherein determining whether the subject is currently in need of rescue intervention includes:
determining that gross motor motion of the subject is present within the extracted motion data; and
determining that the subject is not currently in need of rescue intervention based, at least in part, on the determination that gross motor motion of the subject is present within the extracted motion data.

19. The method of example 17 or example 18 wherein determining whether the subject is currently in need of rescue intervention includes:
determining one or more breathing parameters of the subject based, at least in part, on the extracted motion data;
comparing the one or more breathing parameter of the subject to one or more respective threshold breathing parameters; and
determining that the subject is currently in need of rescue intervention based at least in part on the comparison,
wherein the method further comprises contacting emergency services to rescue the subject, contacting an individual previously specified by the subject to rescue the subject, administering or instructing a device to administer an antidote, or a combination thereof.

20. A method for identifying opioid overdose indicators, the method comprising:
transmitting acoustic energy toward a subject using a first transducer of an electronic device, wherein frequency of the transmitted sound increases from a first frequency to a second frequency over time, and wherein the second frequency is outside of the human audible spectrum of acoustic signals;
acquiring a reflected signal corresponding to the transmitted acoustic energy using a second transducer of the electronic device, wherein the second transducer is configured to produce electrical signals corresponding to the acquired reflected signal;
extracting motion data of the subject from the reflected signal;
determining whether gross motor motion of the subject is present in the extracted motion signal;
when gross motor motion is present in the extracted motion signal, determining that the subject is not currently in need of rescue intervention;
when gross motor motion is not present in the extracted motion signal—
   determining one or more breathing parameters of the subject based at least in part on the extract motion data,
   comparing the one or more breathing parameters to one or more respective threshold breathing parameters, and
   based on the comparison, determining whether the subject is in need of rescue intervention.

21. A non-transitory computer-readable medium having instructions stored thereon that, when executed by an electronic device via one or more processors thereof, cause the electronic device to perform a method for detecting events indicating respiratory failure, the instructions comprising:
instructions to transmit acoustic energy toward a subject using a first transducer of the electronic device, wherein the transmitted acoustic energy increases from a first frequency to a second frequency, and wherein the second frequency is outside of the human audible spectrum of acoustic signals;
instructions to acquire a reflected signal corresponding to the transmitted acoustic energy using a second transducer of the electronic device, wherein the second transducer is configured to produce electrical signals corresponding to the acquired reflected signal;
instructions to identify motion data of the subject in the reflected signal; and
instructions to determine whether the subject is experiencing an event indicating respiratory failure based, at least in part, on the identified motion data.

22. A non-transitory computer-readable medium having instructions stored thereon that, when executed by a mobile device via one or more processors thereof, cause the mobile device to perform a method for identifying opioid overdose indicators, the instructions comprising:
instructions to transmit sound toward a subject using a first transducer of the mobile device, wherein frequency of the transmitted sound increases from a first frequency to a second frequency over time, and wherein the second frequency is outside of the human audible spectrum of acoustic signals;
instructions to acquire a reflected sound signal corresponding to the transmitted sound using a second transducer of the mobile device, wherein the second transducer is configured to produce electrical signals corresponding to the acquired reflected sound signal;
instructions to extract motion data of the subject from the reflected sound signal,
instructions to determine a distance between the subject and the mobile device based at least in part on the reflected sound signal; and
instructions to determine whether the subject is currently in need of rescue intervention based, at least in part, on the extracted motion data.

23. A non-transitory computer-readable medium having instructions stored thereon that, when executed by an electronic device via one or more processors thereof, cause the electronic device to perform a method for identifying opioid overdose indicators, the instructions comprising:
instructions to transmit acoustic energy toward a subject using a first transducer of an electronic device, wherein frequency of the transmitted sound increases from a first frequency to a second frequency over time, and wherein the second frequency is outside of the human audible spectrum of acoustic signals;
instructions to acquire a reflected signal corresponding to the transmitted acoustic energy using a second transducer of the electronic device, wherein the second transducer is configured to produce electrical signals corresponding to the acquired reflected signal;
instructions to extract motion data of the subject from the reflected signal;
instructions to determine whether gross motor motion of the subject is present in the extracted motion signal;
instruction to determine that the subject is not currently in need of rescue intervention when gross motor motion is present in the extracted motion signal;
instructions to determine one or more breathing parameters of the subject based at least in part on the extract motion data when gross motor motion is not present in the extracted motion signal,
instructions to compare the one or more breathing parameters to one or more respective threshold breathing parameters when gross motor motion is not present in the extracted motion signal, and
instruction to determine whether the subject is in need of rescue intervention based on the comparison when gross motor motion is not present in the extracted motion signal.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented and/or discussed in a given order, alternative embodiments can perform steps in a different order. Furthermore, the various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms can also include the plural or singular term, respectively. Furthermore, as used herein, the phrase “and/or” as in “A and/or B” refers to A alone, B alone, and both A and B. Additionally, the terms “comprising,” “including,” “having” and “with” are used throughout to mean including at least the recited feature(s) such that any greater number of the same feature and/or additional types of other features are not precluded.

From the foregoing, it will also be appreciated that various modifications can be made without deviating from the technology. For example, various components of the technology can be further divided into subcomponents, or that various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of operating an electronic device for detecting events indicating respiratory failure, the method comprising:
transmitting acoustic energy toward a subject using a first transducer of the electronic device, wherein the transmitted acoustic energy increases from a first frequency to a second frequency, and wherein the second frequency is outside of the human audible spectrum of acoustic signals;
acquiring a reflected signal corresponding to the transmitted acoustic energy using a second transducer of the electronic device, wherein the second transducer is configured to produce electrical signals corresponding to the acquired reflected signal;
identifying motion data of the subject in the reflected signal; and
determining whether the subject is experiencing an event indicating respiratory failure based, at least in part, on the identified motion data.

2. The method of claim 1 wherein identifying the motion data further includes:
computing a primary transform of the reflected signal;
searching frequency bins of the primary transform for the motion data;
identifying a frequency bin containing the motion data; and
determining a distance between the subject and the second transducer, wherein the distance corresponds to the identified frequency bin.

3. The method of claim 2 wherein searching the frequency bins of the primary transform includes sequentially searching the frequency bins starting with a frequency bin corresponding to a shortest distance from the second transducer and until the motion data is identified in the identified frequency bin.

4. The method of claim 2 wherein the motion data is second motion data, and wherein searching the frequency bins of the primary transform includes searching the frequency bins for the second motion data starting with a previously-identified frequency bin containing first motion data corresponding to a previously acquired portion of the reflected signal.

5. The method of claim 4 wherein the identified frequency bin is different than the previously-identified frequency bin, and wherein determining whether the subject is experiencing an event indicating respiratory failure based, at least in part, on the identified second motion data includes determining the subject is not experiencing an event indicating respiratory failure.

6. The method of claim 2 wherein:
the subject is a first subject, the identified motion data is first identified motion data, the identified frequency bin is a first identified frequency bin, and the distance is a first distance;
the method further comprises identifying second motion data corresponding to a second subject in a second frequency bin of the primary transform of the reflected signal; and
identifying the second motion data includes determining a second distance between the second subject and the second transducer, wherein the second distance corresponds to the second identified frequency bin.

7. The method of claim 6, further comprising determining whether the second subject is experiencing an event indicating respiratory failure based, at least in part, on the second identified motion data.

8. The method of claim 1 wherein determining whether the subject is experiencing an event indicating respiratory failure includes:
determining that gross motor motion of the subject is present in the motion data; and
determining that the subject is not experiencing an event indicating respiratory failure.

9. The method of claim 1 wherein determining whether the subject is experiencing an event indicating respiratory failure includes:
determining one or more breathing parameters of the subject based at least in part on the identified motion data; and
comparing at least one of the one or more breathing parameters to at least one corresponding predetermined threshold.

10. The method of claim 9 wherein determining whether the subject is experiencing an event indicating respiratory failure further includes filtering the motion data to remove frequency higher than 1 Hz.

11. The method of claim 9 wherein determining the one or more breathing parameters includes:
identifying peaks in the identified motion data; and
using at least one of the identified peaks to calculate the one or more breathing parameters, wherein the one or more breathing parameters include a respiratory rate of the subject, an amount of time separating successive peaks identified in the identified motion data, an amplitude of at least one identified peak, a prominence of at least one identified peak, or a combination thereof.

12. The method of claim 11 wherein identifying peaks in the identified motion data includes using previously determined baseline breathing parameters of the subject to identify the peaks, considering only peaks separated by twenty samples of the motion data or more, or a combination thereof.

13. The method of claim 11 wherein:
the one or more breathing parameters include a respiratory rate of the subject, an amount of time separating successive peaks identified in the identified motion data, or a combination thereof;
comparing the one or more breathing parameters include comparing the respiratory rate to a threshold respiratory rate, comparing the amount of time to a threshold amount of time, or a combination thereof;
determining that the respiratory rate of the subject is equal to or less than the threshold respiratory rate, the amount of time separating successive peaks identified in the identified motion data is equal to or greater than the threshold amount of time, or a combination thereof; and
determining whether the subject is experiencing an event indicating respiratory failure further includes determining the subject is experiencing a respiratory depression event, a central apnea event, or a combination thereof.

14. The method of claim 13, further comprising triggering an alert or alarm to prompt the subject for a response.

15. The method of claim 14, further comprising:
determining the subject has not responded to the alert or alarm within a predetermined amount of time, determining the subject is deteriorating based at least in part on a breathing parameter of the subject determined after triggering the alert or alarm, or a combination thereof; and
escalating the alert or alarm by soliciting rescue intervention from emergency services, soliciting rescue intervention from a contact previously specified by the subject, or a combination thereof.

16. The method of claim 14, further comprising:
determining the subject has not responded to the alert or alarm within a predetermined amount of time, determining the subject is deteriorating based at least in part on a breathing parameter of the subject determined after triggering the alert or alarm, or a combination thereof; and
administering or instructing another device to administer an antidote to the subject.

17. A method of operating a mobile device to identify opioid overdose indicators, the method comprising:
transmitting sound toward a subject using a first transducer of the mobile device, wherein frequency of the transmitted sound increases from a first frequency to a second frequency over time, and wherein the second frequency is outside of the human audible spectrum of acoustic signals;
acquiring a reflected sound signal corresponding to the transmitted sound using a second transducer of the mobile device, wherein the second transducer is configured to produce electrical signals corresponding to the acquired reflected sound signal;
extracting motion data of the subject from the reflected sound signal;
determining a distance between the subject and the mobile device based at least in part on the reflected sound signal; and
determining whether the subject is currently in need of rescue intervention based, at least in part, on the extracted motion data.

18. The method of claim 17 wherein determining whether the subject is currently in need of rescue intervention includes:
determining that gross motor motion of the subject is present within the extracted motion data, and
determining that the subject is not currently in need of rescue intervention based, at least in part, on the determination that gross motor motion of the subject is present within the extracted motion data.

19. The method of claim 17 wherein determining whether the subject is currently in need of rescue intervention includes:
determining one or more breathing parameters of the subject based, at least in part, on the extracted motion data;
comparing the one or more breathing parameter of the subject to one or more respective threshold breathing parameters; and
determining that the subject is currently in need of rescue intervention based at least in part on the comparison,
wherein the method further comprises contacting emergency services to rescue the subject, contacting an individual previously specified by the subject to rescue the subject, administering or instructing a device to administer an antidote, or a combination thereof.

20. A method for identifying opioid overdose indicators, the method comprising:
transmitting acoustic energy toward a subject using a first transducer of an electronic device, wherein frequency of the transmitted sound increases from a first frequency to a second frequency over time, and wherein the second frequency is outside of the human audible spectrum of acoustic signals;
acquiring a reflected signal corresponding to the transmitted acoustic energy using a second transducer of the electronic device, wherein the second transducer is configured to produce electrical signals corresponding to the acquired reflected signal;
extracting motion data of the subject from the reflected signal;
determining whether gross motor motion of the subject is present in the extracted motion signal;
when gross motor motion is present in the extracted motion signal, determining that the subject is not currently in need of rescue intervention;
when gross motor motion is not present in the extracted motion signal—
determining one or more breathing parameters of the subject based at least in part on the extract motion data,
comparing the one or more breathing parameters to one or more respective threshold breathing parameters, and
based on the comparison, determining whether the subject is in need of rescue intervention.

21. A non-transitory computer-readable medium having instructions stored thereon that, when executed by an electronic device via one or more processors thereof, cause the electronic device to perform a method for detecting events indicating respiratory failure, the instructions comprising:
instructions to transmit acoustic energy toward a subject using a first transducer of the electronic device, wherein the transmitted acoustic energy increases from a first frequency to a second frequency, and wherein the second frequency is outside of the human audible spectrum of acoustic signals;
instructions to acquire a reflected signal corresponding to the transmitted acoustic energy using a second transducer of the electronic device, wherein the second transducer is configured to produce electrical signals corresponding to the acquired reflected signal;
instructions to identify motion data of the subject in the reflected signal; and
instructions to determine whether the subject is experiencing an event indicating respiratory failure based, at least in part, on the identified motion data.

22. A non-transitory computer-readable medium having instructions stored thereon that, when executed by a mobile device via one or more processors thereof, cause the mobile device to perform a method for identifying opioid overdose indicators, the instructions comprising:
instructions to transmit sound toward a subject using a first transducer of the mobile device, wherein frequency of the transmitted sound increases from a first frequency to a second frequency over time, and wherein the second frequency is outside of the human audible spectrum of acoustic signals;
instructions to acquire a reflected sound signal corresponding to the transmitted sound using a second transducer of the mobile device, wherein the second transducer is configured to produce electrical signals corresponding to the acquired reflected sound signal;
instructions to extract motion data of the subject from the reflected sound signal;
instructions to determine a distance between the subject and the mobile device based at least in part on the reflected sound signal; and
instructions to determine whether the subject is currently in need of rescue intervention based, at least in part, on the extracted motion data.

23. A non-transitory computer-readable medium having instructions stored thereon that, when executed by an electronic device via one or more processors thereof, cause the electronic device to perform a method for identifying opioid overdose indicators, the instructions comprising:
instructions to transmit acoustic energy toward a subject using a first transducer of an electronic device, wherein frequency of the transmitted sound increases from a first frequency to a second frequency over time, and wherein the second frequency is outside of the human audible spectrum of acoustic signals;
instructions to acquire a reflected signal corresponding to the transmitted acoustic energy using a second transducer of the electronic device, wherein the second transducer is configured to produce electrical signals corresponding to the acquired reflected signal;
instructions to extract motion data of the subject from the reflected signal;
instructions to determine whether gross motor motion of the subject is present in the extracted motion signal;
instruction to determine that the subject is not currently in need of rescue intervention when gross motor motion is present in the extracted motion signal;
instructions to determine one or more breathing parameters of the subject based at least in part on the extract motion data when gross motor motion is not present in the extracted motion signal,
instructions to compare the one or more breathing parameters to one or more respective threshold breathing parameters when gross motor motion is not present in the extracted motion signal, and
instruction to determine whether the subject is in need of rescue intervention based on the comparison when gross motor motion is not present in the extracted motion signal.
